(19) United States
(12) Patent Application Publication
(10) Pub. No.: US 2008/0245890 A1
(43) Pub. Date: Oct. 9, 2008

(54) Device for Dispensing Volatile Compounds in a Gel to the Ambient Air

(75) Inventors: Derek Alan Lockwood, Auckland (NZ); Neil Campbell Muir, Dansom Lane (GB)

Correspondence Address: NORRIS, MCLAUGHLIN & MARCUS, 875 THIRD AVE, 18TH FLOOR, NEW YORK, NY, 10022, US

(73) Assignee: Reckitt Benckiser (UK) Limited, Slough, Berkshire (GB)

(21) Appl. No.: 10/571,433

(22) PCT Filed: Sep. 17, 2004
(86) PCT No.: PCT/GB04/03974

§ 371 Date: Mar. 13, 2006

(30) Foreign Application Priority Data
Sep. 19, 2003 (GB) 0321938.3

(51) Int. Cl. A61L 9/04; C11D 3/50

(52) U.S. Cl. 239/60; 523/102

(57) ABSTRACT

The invention relates to improvements in or relating to containers and in particular to an air freshening or purifying device comprising a primary container comprising an inner section and outer section each having a circumferential side wall. One of the sections has retaining means which defines at least one vapour release aperture and the other having a base. The inner section side wall is dimensioned so as to fit inside the outer section side wall, the two sections being interlockable to form the primary container. A refill container has a gel receiving surface having at least one recess for receiving a gel composition, and is located and firmly held within the primary container, the base being provided with means for attachment of the device to a surface.

The invention relates to improvements in or relating to containers and in particular to an air freshening or purifying device utilising a gel fragrance or other gel composition comprising a base container for receiving a refill containing the composition.

U.S. Pat. No. 5,780,527 describes a gel which can be used as a fragrancing component in an air freshening device. This gel is particularly advantageous in that it can be used in attractively shaped open containers without the need for sealing. One air freshening device which is currently on the market comprises an attractive glass open sided container, which is recessed to form a dish with a base and circumferential side wall. The dish stands upright on a flattened section of its perimeter. A plurality of ridges are provided on the inner surface of the container base defining channels between the ridges, in which the gel is retained. As the fragrance is dissipated over time, the gel shrinks and cracks and is no longer wholly supported by the ridge walls. To prevent the shrinking gel from falling out of the container, a number of smallish channels are used, which are fairly narrow or have narrow sections.

It is desirous for the consumer to have a means of refilling the container once the gel fragrance has dissipated. However, as the filling process comprises the steps of filling the channels with the gel in liquid form and allowing the gel to set, this is not a process which the consumer is able to carry out. A refillable version of this air freshening device is described in GB-A2374805 which comprises a primary container having a gel receiving surface having recesses for receiving a gel composition, and a refill container having a gel receiving surface profiled to correspond to the gel receiving surface of the primary container and also having recesses for receiving the gel. The refill container has an opposing rear surface, the profile of which inversely corresponds to the gel receiving surface. of the primary container and is dimensioned so as to abut closely with and interlock with the gel receiving surface of the primary container so as to be retained thereby.

The gel composition is a fragrance or air purifying composition, or an insecticide. The gel may be as described in, for example, U.S. Pat. No. 5,780,527. Thus it is, for example, a gel resulting from the cross-linking, in situ, of a homopolymer or copolymer in the presence of a perfuming, deodorizing or insecticidal base. A suitable copolymer is maleinised polybutadiene or polyisoprene such as Lithene N4-9000 10MA (Registered Trade Mark) obtainable from Revertex Ltd. A suitable cross-linking agent, for example, a diamine, being a low molecular weight “polymer” containing two amine groups per molecule sold under the name Jeffamine 400 (Registered Trade Mark) obtainable from Huntsman Corp.

Whilst this air freshening device has been very successful, it can only be used in a free standing mode, where there is a suitably flat surface to place it on. It is desirable to have an air freshening device, which comprises a primary container, and a secondary refill container, which can be used in a variety of locations, such as being attached to a door or wall, used in a car and so on.

It is an object of the present invention to provide such a versatile air freshening device.

According to the invention there is therefore provided an air freshening or purifying device comprising a primary container comprising an inner section and an outer section, each section having a circumferential side wall, one of the sections having retaining means which defines at least one vapour release aperture and the other having a base, the inner section side wall being dimensioned so as to fit inside the outer section side wall, the two sections being interlockable to form the primary container, the air freshening or purifying device further comprising a refill container having a gel receiving surface having at least one recess for receiving a gel composition, which refill container is located and firmly held within the primary container, the base being provided with means for attachment of the device to a surface.

A preferred embodiment of the present invention will now be described, by way of example only, with reference to the accompanying drawings in which:

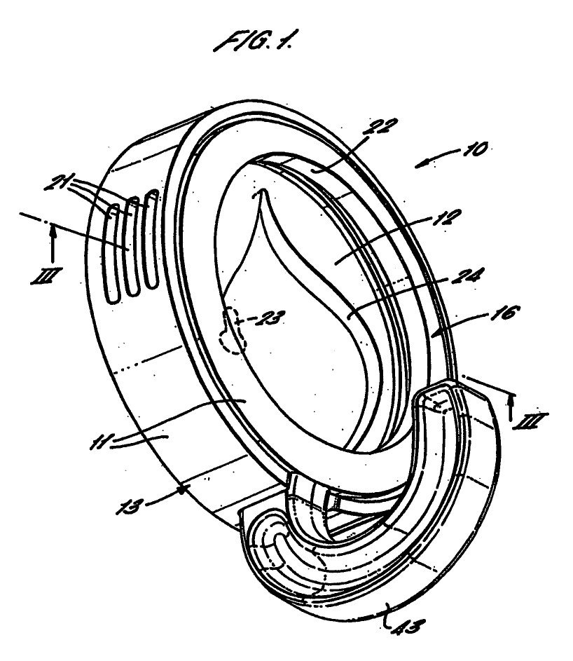

FIG. 1 is a pictorial representation of an air freshening device of the present invention with a refill container loaded into the primary container, having a hook attachment;

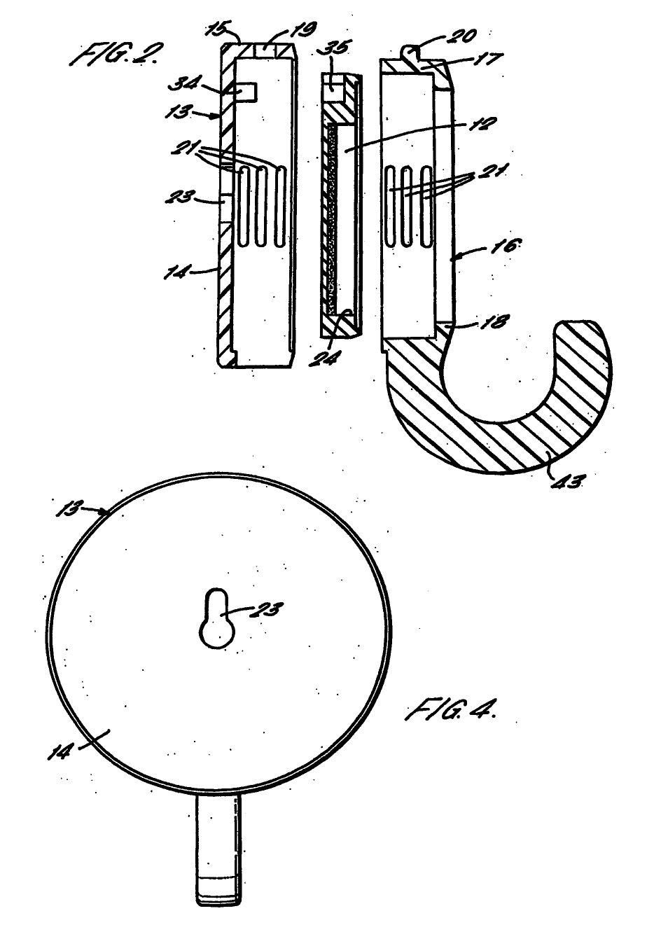

FIG. 2 is an exploded cross-sectional side elevation of the air freshening device of FIG. 1 on the line II-II showing the two parts of the primary container separated and the refill container between them;

FIG. 3 is a cross-sectional plan view of the device of FIG. 1 on the line III-III;

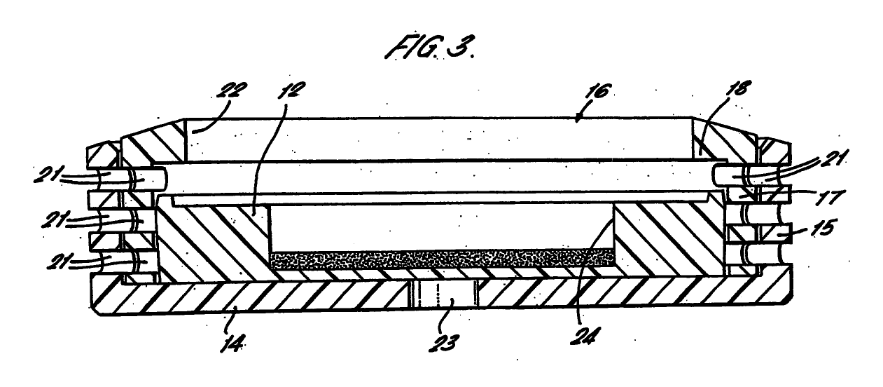

FIG. 4 is a rear elevation of the air freshening device of FIG. 1;

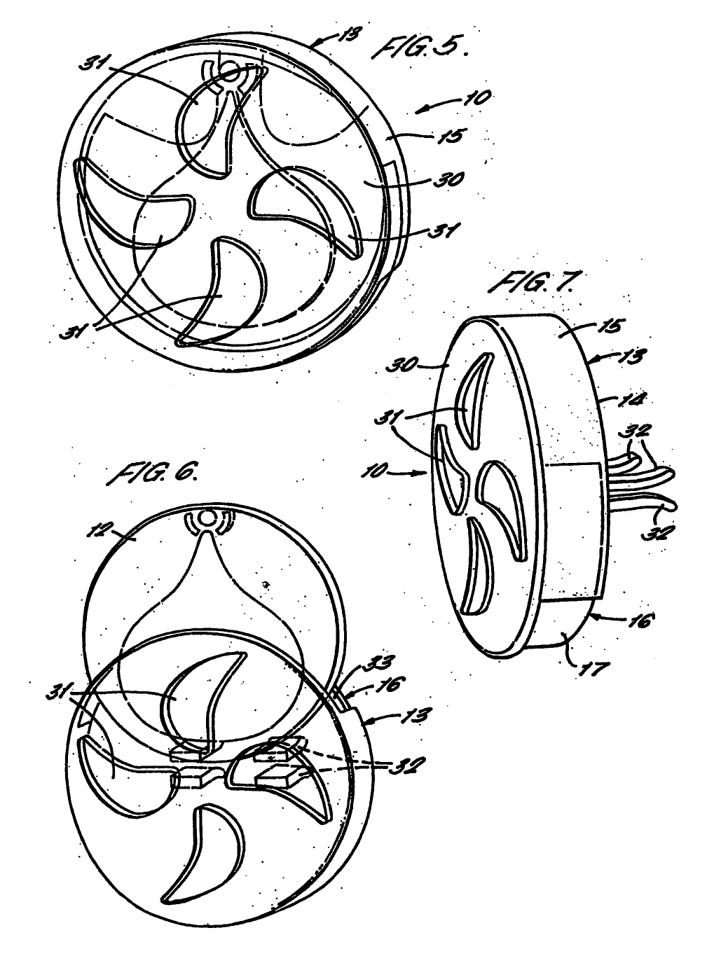

FIGS. 5 and 6 are front elevations of an alternative embodiment of an air freshening device according to the invention, intended for attachment to an air vent;

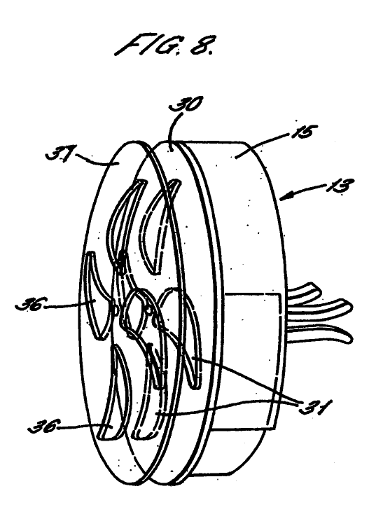

FIG. 7 is a side elevation of the device of FIG. 5;

FIG. 8 is a side elevation of an alternative embodiment of the device of FIG. 5;

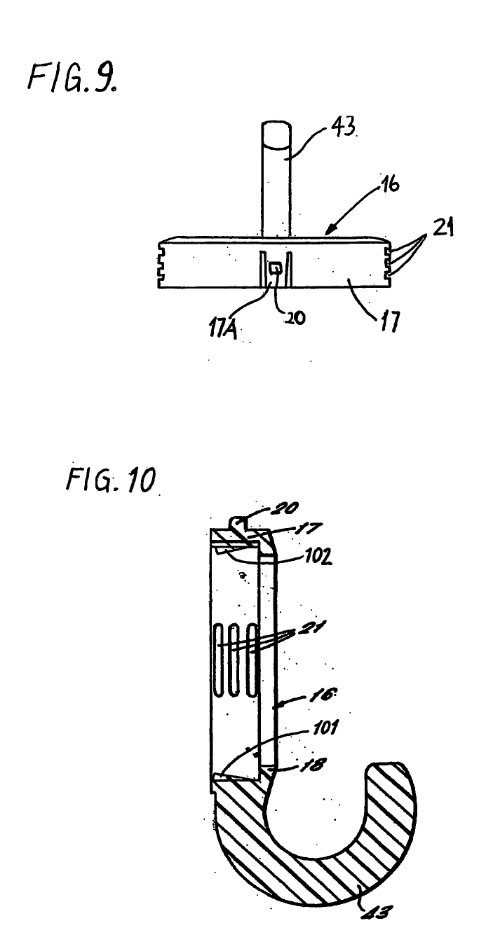

FIG. 9 is a top view of the secondary container; and

FIG. 10 is a cross sectional side elevation of an alternative embodiment of the secondary container shown in FIG. 2.

Referring to FIGS. 1 and 2 there is shown an air freshening (or purifying) device 10. The device 10 comprises a primary container 11 and a refill container 12. The primary container 11 comprises two sections, a first (outer) section 13 having a base 14 and a circumferential side wall 15. The second (inner) section 16 also has a circumferential side wall 17, which is shaped and dimensioned so as to fit snugly inside the side wall 15 of the primary container 11. The inner section 16 also has a flange 18 extending inwardly from the side wall 17 defining an aperture 22. The air freshening or purifying vapour leaves the device via this aperture 22.

The inner and outer sections 13, 16 are provided with co-operating locking means, see FIG. 2, to enable them to be secured together. In the embodiment illustrated, the locking means comprise a hole 19 in the side wall 15 of the first (outer) section 13 for receiving a small spigot 20 on the external surface of the side wall 17 of the second (inner section). When the spigot 20 is inserted into the hole 19, and the sections 13, 16 are squeezed together, they snap into a closed position, as shown in FIG. 1, (thereby forming the primary container 11). The spigot 20 is preferably mounted on a hinged section 17A of the sidewall 17, which can move inwardly relate to the rest of the sidewall 17. This means it is easier to connect to locking means (see FIG. 9).

The side walls 15, 17 are preferably provided with air vents 21, which align with each other when the primary container 11 is in its closed position, see FIG. 3. The air vents 21 may comprise a series of slots as shown, or they may be circular or other shaped apertures The air vents 21 also allow the air freshening or purifying vapour to be released into the atmosphere.

The primary container 11 may be provided with additional features to give it a secondary purpose. In the embodiment illustrated in FIG. 1, for example, the inner section 16 is provided with a hook 43. In the base 14 of the outer section 13 is provided a slot 23 having a narrow end and an enlarged end, for receiving the head of a screw or nail, see FIG. 4. The air freshening device 10 can be attached to a screw or nail (located in a wall, door or the like), by placing the enlarged end of the slot 23 over the screw/nail head, and sliding the device 10 downwards so that the screw/nail body slides into to narrow end of the slot 23, so that the screw/nail head is held firmly inside the outer section 13. The air freshening device 10 can then also be used as a hanger on which to hang things.

As an alternative to the slot 23, a double sided sticky pad or adhesive base may be applied to the base 14 of the outer section 11, so that the device 10 may be stuck onto a convenient surface, which may be at any angle.

The refill container 12 is dimensioned so as to have at least one diameter greater than the largest diameter of the flange aperture 22. The refill container 12 is placed between the two primary container sections 13,16 before they are closed together, and will be held securely within the primary container 11 as the flange 18 provides a retaining means which prevents the refill container 12 from falling out. Both the width and the depth of the refill container 12 are selected so that the refill container 12 does not rattle or move very much, when located inside the closed primary container 11.

The refill container 12 and primary container 11 may also be provided with locating means, which enable the refill container 12 to be held in a particular orientation within to primary container 11. This is preferable where the gel receiving surface is of a particular shape where it is preferred that it be oriented in a particular manner. In one embodiment the locating means are provided by a small projection 34 on the base 14, see FIG. 2, which is inserted into a small hole 35 in the refill container 12. Other arrangements are also suitable, see FIGS. 9 and 10.

As shown in FIG. 9, the spigot 20 can be mounted on a cut away section 17A of the side wall 17 to render it hinged and therefore capable of flexible movement, as described above.

In FIG. 10 it is shown in an alternative embodiment that locating means 101 and 102 are located on an inner section of the side wall 17. The locating means 101 and 102 are dimensioned to co-operate with matching features on a refill to locate the refill in a particular orientation within the primary container 11. The locating means are also tapered so as to bias the refill into engagement with the second inner section 16. In this embodiment the locating means 101 and 102 are diametrically opposed on the inner section of the side wall.

In the embodiment of the invention illustrated in FIGS. 5, 6 and 7, the air freshening device 10 is intended for attachment to an air vent for example in an air conditioning unit in an office or domestic environment, or in a vehicle. It also incorporates a slightly modified structure of the base container 11, which can also be used in other applications too.

In this embodiment the outer section 13 has a circumferential side wall 15, and a fascia 30, which replaces the flange 18, as the means for retaining the refill container 12 within the primary container 11. The fascia 30 is provided with a plurality of apertures 31, which replaces the single aperture 22, for allowing the dissipation of the air freshening or purifying vapour. The inner section 16 also has a circumferential side wall 17 and further provides the base 14, to which are attached a plurality of attachment fingers 32. The attachment fingers 33 are designed to attach to the bars of an air vent.

The base 14 also has apertures which enable to air from the vent to enter the primary container 11 and flow over the gel in the refill container 12. The vapour is picked up in the air flow by the venturi effect. As before, the side walls 15, 17 are dimensioned so as to fit one inside the other and snap together to remain firmly closed. However, in this embodiment the inner and outer sections 13, 16 are rotatable relative to each other. A section of each side wall 15, 17 is missing, which means that the inner and outer sections 13, 16 can be rotated to line up the missing sections to provide an opening 33. The opening 33 is of a size to enable the refill container 12 to be inserted into the primary container 11. When the missing sections are moved out of alignment, the refill container 12 is retained securely within.

As shown in FIG. 8 means may also be provided to enable the apertures 31 to be wholly or partly closed to restrict the airflow through the primary container 11, to thereby control the release of the vapour into the atmosphere. In this embodiment the airflow restriction means comprise a disc 37, the shape of which preferably corresponds to the fascia 30. The disc 37 is provided with apertures 36 of similar, preferably indentical, shape and size to the apertures 31 in the fascia 30. The disc 37 is co-axially and rotatably mounted to the fascia 30. As such the device 10 can be opened, with the apertures 31,36 aligned allowing air flow therethrough. The Device 10 can also be closed by moving the apertures 31, 36 totally out of alignment, thereby occluding air flow. A restricted airflow can also be provided by particularly overlapping the apertures 31,36.

Although the containers 11 and 12 illustrated are substantially circular, other shapes can easily be used. The primary container 11 may be made from clear, translucent and/or coloured plastic, although other suitable reasonably rigid impermeable materials could be used.

The refill container 12 has a gel receiving surface in which is provided one or more recesses 24. The profile of the recesses 24 is not limited to that shown. There may be a plurality of narrow recesses 24 separated by ridges, whereby the recesses 24 are reasonably narrow in this embodiment to hold the gel where it shrinks or cracks, such as is illustrated in GB-A-2374805. Alternatively, where there is only a single or a few larger recesses 24 additional means are provided to help hold the gel in the recesses 24. These may comprise projections located on the base of the recess(es) 24, such as are described in GB-A-2370990. The recess(es) 24 preferably provide an attractive pattern, so that when the refill container 12 is filled with a gel composition, preferably of the type described in U.S. Pat. No. 5,780,527, which is preferably strongly coloured, the shape of the recess(es) 24 is highlighted to give an attractive appearance.

The refill container 12 is preferably made of glass or a transparent plastic or other impermeable material. Prime examples of suitable materials are APET, PETG, Polypropylene and Polyacrylonitrile as these have a high degree of clarity, are easy to thermoform and are resistant to attack by perfume. Further materials may comprise Polyethylene and Nylon, although these tend to be translucent or of a milky appearance, or PVC, Polystyrene and Styrene-Acrylonitrile, although these may be susceptible to fragrance attack.

The refill container 12 may also be translucent and/or coloured, for use with an air freshening device 10 which has a primary container 11 which is translucent and/or coloured. The colour used may be suggestive of the fragrance of the gel composition, e.g. yellow for lemon, pink for rose etc.

The relative sizing of the containers 11, 12 ensures that the refill container 15 is held firmly in position by the primary container 12, although a releasable adhesive could be used to ensure this.

Once the gel composition in the refill container 12 has dissipated over time, the refill container 12 can simply be removed from container and a new one inserted.

The refill container 12 is manufactured by a suitable method, such as vacuum forming, thermoforming or injection moulding. The recess(es) 24 is (are) then filled with the gel composition in liquid form and the gel allowed to set.

The refills can conveniently be provided with a removable lid, in the form of a tear off plastic or foil cover, to protect the gel before use. The shape of the refills also allows them to conveniently be stacked, so that a number can be sold together in a tube packaging.

The refill may be filled with a gel fragrance, which would provide as air freshening action, or with other gel based air purifying compositions, such as insecticides or disinfectants. Thus one primary container 11 could be used for a variety of different applications, merely by changing the refill container 12 filled with the required composition.

1. An air freshening or purifying device comprising a primary container comprising an inner section and an outer section, each section having a circumferential side wall, one of the sections having retaining means which defines at least one vapour release aperture and the other having a base, the inner section side wall being dimensioned so as to fit inside the outer section side wall, the two sections being interlockable to form the primary container, the air freshening or purifying device further comprising a refill container having a gel receiving surface having at least one recess for receiving a gel composition, which refill container is located and firmly held within the primary container, the base being provided with means for attachment of the device to a surface.

2. A device according to in claim 1 in which the retaining means comprises a flange defining the at least one vapour release aperture, which flange projects inwardly from the side wall of one of the sections.

3. A device according to claim 1 in which the retaining means comprises a fascia defining the at least one vapour release aperture.

4. A device according to claim 3 in which the retaining means comprises a fascia defining a plurality of vapour release apertures.

5. A device according to claim 1 in which locking means are provided to lock together the two sections of the primary container.

6. A device according to claim 5 in which the locking means comprise an aperture in one of the section side walls for receiving a spigot mounted on the other of the section side walls.

7. A device according to claim 6 in which the spigot is mounted on a portion of one of the section side walls which is hinged and moveable inwardly relative to the rest of the said side wall.

8. A device according to claim 1 in which each section side wall is provided with at least one air vent, the vent(s)in the inner section side wall being alignable with the vent(s) in the outer section side wall to provide further vapour release.

9. A device according to claim 1 in which the primary container is provided with a hook.

10. A device according to claim 1 in which the means for attachment comprise a slot in the base of the one section, said slot having an enlarged end and a narrow end.

11. A device according to claim 1 in which the means for attachment comprise an adhesive layer on the base of the one section.

12. A device according to claim 1 in which the means for attachment comprise a plurality of fingers projecting from the base of the one section.

13. A device according to claim 1 in which the refill container has at least one diameter which is greater than the largest diameter of the at least one vapour release aperture defined by the retaining means.

14. A device according to claim 1 in which the refill container diameter(s) and depth are selected so as to be smaller, but not substantially smaller, than inside diameter(s) and depth of the primary container.

15. A device according to claim 1 further comprising locating means for orienting and securing the refill container in a particular orientation.

16. A device according to claim 15 in which the locating means comprise a projection on the base of the one section or one of the section side walls, and a aperture on the refill container for receiving said projection.

17. A device according to claim 1 in which the inner and outer sections are rotatable relative to each other.

18. A device according to claim 17 in which a portion of each section side wall is removed to form gaps, which gaps are alignable with each other on rotation of the sections relative to each other to define an opening for receiving the refill container.

19. A device according to claim 1 in which the size of the vapour release aperture(s) defined by the restraining means can be varied, to control air flow therethrough.

20. A device according to claim 1 in which the refill container is transparent, translucent or coloured.

21. (canceled)

22. (canceled)

23. A device according to claim 1 in which the gel receiving surface is provided with at least on gel receiving recess.

24. A device according to claim 23 in which the gel receiving surface comprises a plurality of recesses separated by ridges.

25. A device according to claim 1 in which the gel composition is a fragrance or an insecticide.

26. (canceled)

27. (canceled)
